(19) United States
(12) Patent Application Publication
(10) Pub. No.: US 2011/0256601 A1
(43) Pub. Date: Oct. 20, 2011

(54) MODIFICATION OF HYDROGENASE ACTIVITIES IN THERMOPHILIC BACTERIA TO ENHANCE ETHANOL PRODUCTION

(75) Inventors: Arthur Josephus Shaw, IV, West Lebanon, NH (US); Lee Lynd, Meriden, NH (US); David A. Hogsett, Grantham, NH (US)

(73) Assignee: THE TRUSTEES OF DARTMOUTH COLLEGE, Hanover, NH (US)

(21) Appl. No.: 12/808,764

(22) PCT Filed: Dec. 17, 2008
(86) PCT No.: PCT/US08/87235

§ 371 Date: Jul. 6, 2011

Related U.S. Application Data
(60) Provisional application No. 61/014,359, filed on Dec. 17, 2007.
(60) Provisional application No. 61/049,238, filed on Apr. 30, 2008.

(51) Int. Cl. C12P 7/10; C12N 1/19; C12N 9/02; C12P 7/06; C12N 1/20; C07H 21/04; C12N 1/21; C12N 15/63

(52) U.S. Cl. 435/165; 435/252.3; 435/254.2; 435/320.1; 435/161; 435/252.1; 536/23.7; 435/189

(57) ABSTRACT

Bacteria consume a variety of biomass-derived substrates and produce ethanol. Hydrogenase genes have been inactivated m Thermoanaerobacterium saccharolyticum to generate mutant strains with reduced hydrogenase activities. One such mutant strain with both the ldh and hydtrA genes inactivated shows a significant increase in ethanol production. Manipulation of hydrogenase activities provides a new approach for enhancing substrate utilization and ethanol production by biomass-fermenting microorganisms.

### RELATED APPLICATIONS

This application claims priority to U.S. Provisional Application No. 61/014,359, filed Dec. 17, 2007, and U.S. Provisional Application No. 61/049,238, filed Apr. 30, 2008, each of which is incorporated herein by reference.

### GOVERNMENT INTERESTS

The United States Government may have certain rights in this invention as research relevant to its development was funded by National Institute of Standards and Technology (NIST) contract number 60NANB1D0064.

### BACKGROUND

1. Field of the Invention

The present invention pertains to the field of biomass processing to produce ethanol. In particular, new thermophilic organisms that can use a variety of biomass derived substrates and produce ethanol in high yield are disclosed.

2. Description of the Related Art

Lignocellulosic biomass represents one of the most abundant renewable resources on Earth. It is formed of three major components—cellulose, hemicellulose, and lignin—and includes, for example, agricultural and forestry residues, municipal solid waste (MSW), fiber resulting from grain operations, waste cellulosics (e.g., paper and pulp operations), and energy crops. The cellulose and hemicellulose polymers of biomass may be hydrolyzed into their component sugars, such as glucose and xylose, which can then be fermented by microorganisms to produce ethanol. Conversion of even a small portion of the available biomass into ethanol could substantially reduce current gasoline consumption and dependence on petroleum.

Significant research has been performed in the areas of reactor design, pretreatment protocols and separation technologies, so that bioconversion processes are becoming economically competitive with petroleum fuel technologies. However, it is estimated that the largest cost savings may be achieved by combining two or more process steps. For example, simultaneous saccharification and fermentation (SSF) and simultaneous saccharification and co-fermentation (SSCF) processes combine an enzymatic saccharification step with fermentation in a single reactor or continuous process apparatus. In an SSF process, end-product inhibition is removed as the soluble sugars are continually fermented into ethanol. When multiple sugar types are fermented by the same organism, the SSF process is usually referred to as a simultaneous saccharification and co-fermentation (SSCF) process.

In addition to savings associated with shorter reaction times and reduced capital costs, co-fermentation processes may also provide improved product yields because certain compounds that would otherwise accrue at levels that inhibit metabolysis or hydrolysis are consumed by the co-fermenting organism(s). In one such example, β-glucosidase ceases to hydrolyze cellobiose in the presence of glucose and, in turn, the build-up of cellobiose impedes cellulose degradation. An SSCF process involving co-fermentation of cellulose and hemicellulose hydrolysis products may alleviate this problem by converting glucose into one or more products that do not inhibit the hydrolytic activity of β-glucosidase.

Consolidated bioprocessing (CBP) involves four biologically-mediated events: (1) enzyme production, (2) substrate hydrolysis, (3) hexose fermentation and (4) pentose fermentation. In contrast to conventional approaches, which perform each step independently, all four events may be performed simultaneously in a CBP configuration. This strategy requires a microorganism that utilizes both cellulose and hemicellulose. Otherwise, a CBP process that utilizes more than one organism to accomplish the four biologically-mediated events is referred to as a consolidated bioprocessing co-culture fermentation.

In SSF, SSCF and CBP processes, bacterial strains that have the ability to convert pentose sugars into hexose sugars, and to ferment the hexose sugars into a mixture of organic acids and other products via glycolysis perform a crucial function. The glycolytic pathway begins with conversion of a six-carbon glucose molecule into two three-carbon molecules of pyruvate. Pyruvate may then be converted to lactate by the action of lactate dehydrogenase (“ldh”), or to acetyl coenzyme A (“acetyl-CoA”) by the action of pyruvate dehydrogenase or pyruvate-ferredoxin oxidoreductase. Acetyl-CoA is further converted to acetate by phosphotransacetylase (“pta”) and acetate kinase (“ack”), or reduced to ethanol by acetaldehyde dehydrogenase (“AcDH”) and alcohol dehydrogenase (“adh”).

Carbohydrate metabolic pathways, such as those described above, may be altered by directing the flow of carbon to a desired end product, such as ethanol. See generally, Lynd, L. R., P. J. Weimer, W. H. van Zyl, and I. S. Pretorius (2002) Microbial cellulose utilization: Fundamentals and biotechnology. Microbiol. Mol. Biol. Rev. 66: 506. A “carbon-centered” approach to metabolic engineering involves inactivating enzymatic pathways that direct carbon containing molecules away from ethanol or otherwise promoting the flow of carbon towards ethanol. For instance, Desai, S. G., M. L. Guerinot, L. R. Lynd (2002) Cloning of L-lactate dehydrogenase and elimination of lactic acid production via gene knockout in Thermoanaerobacterium saccharolyticum JW/SL-YS485. Appl. Microbiol. Biotechnol. 65: 600-605 and PCT/US07/67941, describe the inactivation of L-lactate dehydrogenase (ldh) alone and in combination with acetate kinase (ack) and/or phosphotransacetylase (pta), respectively, which results in strains that produce ethanol in higher yields than native organisms.

Although a “carbon-centered” approach to producing knockout organisms represents an advance in the art, additional and/or alternative approaches to modifying the glycolytic pathway may result in more efficient biomass conversion.

### SUMMARY

The present instrumentalities advance the art by providing methods for manipulating branched end-product metabolism of fermentative microorganisms. The relative production of solvents to organic acids is changed by virtue of eliminating one or more enzyme activities associated with the formation of hydrogen. More specifically, the present instrumentalities advance the art by providing bacteria with mutation in their hydrogenase genes. Such organisms may utilize a variety of biomass derived substrates to generate ethanol in high yields. Methods for generating such organisms by genetic engineering are also disclosed.

The instrumentalities reported herein result in the knockout of various genes either singly or in combination, where such genes in the native organism would otherwise result in the formation of hydrogen and organic acids. These knockout organisms may include but are not limited to those where the following genes are disrupted: (a) hyd hydrogenase, (b) hydtr hydrogenase, (c) hyd and hydtr hydrogenases, and (d) hyd and/or hydtr hydrogenases with one or more of acetate kinase (ack), phosphotransacetylase (pta) and lactate dehydrogenase (ldh).

In an embodiment, an organism having at least one hydrogenase gene that is endogenous to the organism which has been inactivated by genetic engineering is capable of fermenting a saccharification product derived from a carbohydrate-rich biomass substrate.

In an embodiment, a bacterium having ldh and hydtrA genes that are inactivated by genetic engineering is capable of fermenting a saccharification product derived from a carbohydrate-rich biomass substrate.

In an embodiment, a bacterium having at least one hydrogenase gene that is endogenous to the bacterium which has been inactivated by genetic engineering is capable of fermenting a saccharification product derived from a carbohydrate-rich biomass substrate.

In an embodiment, a Thermoanaerobacterium saccharolyticum strain deposited under Patent Deposit Designation No. PTA-8897 is described.

In an embodiment, an isolated polynucleotide comprising a nucleotide sequence having at least 90% sequence identity with a polynucleotide sequence selected from the group consisting of SEQ ID NOS: 1-8 is described.

In an embodiment, an isolated polynucleotide molecule comprising a polynucleotide sequence selected from the group consisting of SEQ ID NOS: 1-8 is described.

In an embodiment, a genetically engineered cell expressing a hydrogenase encoded by a gene having at least 90% sequence identity with a nucleotide sequence selected from the group consisting of SEQ ID NOS: 1-8, the expression of said hydrogenase being driven by a heterologous promoter, is described.

In an embodiment, a genetic construct comprising a coding sequence having at least 90% sequence identity with a nucleotide sequence selected from the group consisting of SEQ ID NOS: 1-8, said coding sequence being operably linked to a promoter capable of controlling transcription in a bacterial cell, is described.

In an embodiment, a method for producing ethanol includes generating an organism with at least one hydrogenase gene inactivated, and incubating the organism in a medium containing at least one substrate selected from the group consisting of glucose, xylose, mannose, arabinose, galactose, fructose, cellobiose, sucrose, maltose, xylan, mannan, starch, cellulose, pectin and combinations thereof to allow for production of ethanol from the substrate.

In an embodiment, a method for producing ethanol includes providing within a reaction vessel, a reaction mixture comprising a carbohydrate-rich biomass substrate, a cellulolytic material, and a fermentation agent, the fermentation agent comprising a bacterium that has been genetically modified to inactivate at least one hydrogenase gene that is endogenous to said bacterium, where the reaction mixture is incubated under suitable conditions for a period of time sufficient to allow saccharification and fermentation of the carbohydrate-rich biomass substrate.

In an embodiment, an isolated protein molecule having hydrogenase activity and comprising a polypeptide having an amino acid sequence having at least 90% sequence identity with a polypeptide selected from the group consisting of SEQ ID NOS: 9-16 is described.

In an embodiment, a bacterium having at least one hydrogenase gene that is endogenous to the bacterium which has been inactivated by genetic engineering is capable of fermenting a saccharification product derived from a carbohydrate-rich biomass substrate.

### BRIEF DESCRIPTION OF THE DRAWINGS

FIG. 1 shows a modified glycolytic pathway after hydrogenase inactivation, according to an embodiment.

FIG. 2 shows the genomic structure of the hyd operon, according to an embodiment.

FIG. 3 shows the genomic structure of the hydtr operon, according to an embodiment.

### DETAILED DESCRIPTION

There will now be shown and described methods for engineering and utilizing thermophilic, anaerobic, Gram-positive bacteria in the conversion of biomass to ethanol.

As used herein, an organism is in “a native state” if it has not been genetically engineered or otherwise manipulated by the hand of man in a manner that alters the genotype and/or phenotype of the organism. For example, a wild-type organism may be considered to be in a native state.

“Identity” refers to a comparison between sequences of polynucleotide or polypeptide molecules. Methods for determining sequence identity are commonly known. Computer programs typically employed for performing an identity comparison include, for example, the Gap program (Wisconsin Sequence Analysis Package, Version 8 for Unix, Genetics Computer Group, University Research Park, Madison Wis.), which uses the algorithm of Smith and Waterman (1981) Adv. Appl. Math. 2: 482-489.

“Lignocellulosic substrate” generally refers to any lignocellulosic biomass suitable for use as a substrate to be converted into ethanol.

“Saccharification” refers to the process of breaking a complex carbohydrate, such as starch or cellulose, into its monosaccharide or oligosaccharide components. For purposes of this disclosure, a complex carbohydrate is preferably processed into its monosaccharide components during a saccharification process.

The teem “endogenous” is used to describe a molecule that exists naturally in an organism. A molecule that is introduced into an organism using molecular biology tools, such as transgenic techniques, is not endogenous to that organism.

The terms “inactivated”, “inactivate”, or “gene inactivation” refer to a process by which a gene is rendered substantially non-expressing and/or non-functional. The teen “substantially” means more than seventy percent. Thus, for purposes of this disclosure, a gene is considered inactivated if its expression or its function has been reduced by more than seventy percent. Techniques for inactivation of a gene may include, but are not limited to, deletion, insertion, substitution in the coding or non-coding regulatory sequences of the target gene, as well as the use of RNA interference to suppress gene expression. The process of inactivating a gene is frequently referred to as “knocking out” a gene. Thus, an organism that has one or more of its genes inactivated may be called a “knockout” (KO) strain.

For purposes of this disclosure, an organism that possesses the necessary biological and chemical components, including polynucleotides, polypeptides, carbohydrates, lipids and other molecules, as well as cellular or subcellular structures that may be required for performing or facilitating certain biological and/or chemical processes is deemed to be capable of performing said processes. Thus, an organism that contains certain inducible genes may be considered capable of performing the function attributable to the protein encoded by those genes.

The term “genetic engineering” is used to refer to a process by which genetic materials, including DNA and/or RNA, are manipulated in a cell or introduced into a cell to affect expression of certain proteins in said cell. Manipulation may include introduction of a foreign (or “exogenous”) gene into the cell or inactivation or modification of an endogenous gene. Such a modified cell may be called a “genetically engineered cell” or a “genetically modified cell”. If the original cell to be genetically engineered is a bacterial cell, said genetically engineered cell may be said to have been derived from a bacterial cell. A molecule that is introduced into a cell to genetically modify the cell may be called a genetic construct. A genetic construct typically carries one or more DNA or RNA sequences on a single molecule.

The expression of a protein is generally regulated by a non-coding region of a gene termed a promoter. When a promoter controls the transcription of a gene, it can also be said that the expression of the gene (or the encoded protein) is driven by the promoter. When a promoter is placed in proximity of a coding sequence, such that transcription of the coding sequence is under control of the promoter, it can be said that the coding sequence is operably linked to the promoter. A promoter that is not normally associated with a gene is called a heterologous promoter.

A “cellulolytic material” is a material that may facilitate the breakdown of cellulose into its component oligosaccharides or monosaccharides. For example, cellulolytic material may comprise a cellulase or hemicellulase.

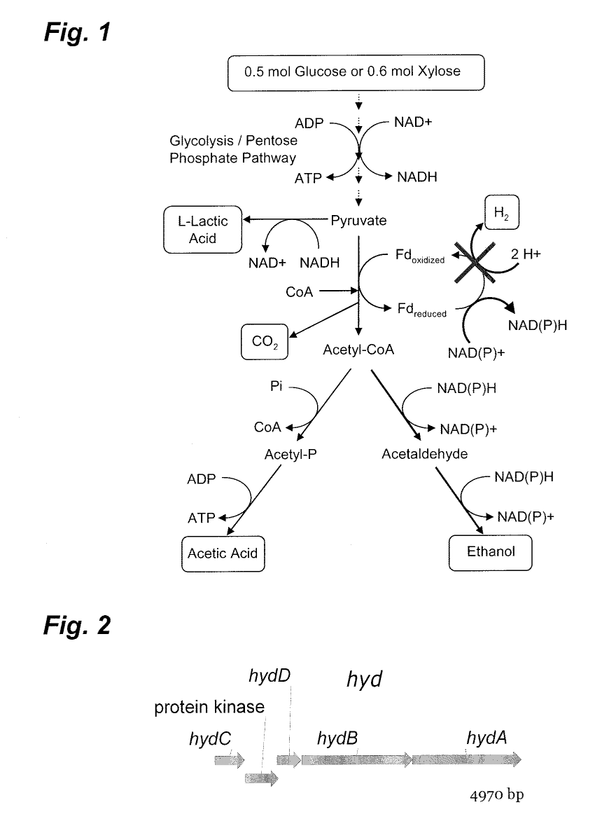

As discussed above, carbohydrate metabolic pathways in a microorganism may be altered by directing the flow of carbon to a desired end product, such as ethanol, using a “carbon-centered” approach to metabolic engineering. An alternative, “electron-centered” approach, is disclosed herein where ethanol yield may be increased by inactivation of an enzymatic pathway that produces hydrogen. For example, FIG. 1 illustrates a portion of the glycolytic pathway, where a cross indicates blocking of hydrogenase activity that leads to hydrogen production. Based on stoichiometric equations, it has been shown that hydrogen production is related to acetic acid production. Therefore, disrupting the ability of an organism to produce hydrogen results in decreased production of acetic acid and increased ethanol production.

The vast majority of high yield ethanol producing microorganisms use a key enzyme, pyruvate decarboxylase (PDC), to form ethanol. In contrast, engineered strains of T. saccharolyticum disclosed herein use a series of enzymes, pyruvate:ferredoxin oxidoreductase, ferredoxin:NADH oxidoreductase, and acetaldehyde dehydrogenase to perform the same molecular rearrangement as PDC. In native non-engineered strains of T. saccharolyticum, only a fraction of the total metabolic flux passes through these enzymes and subsequently to ethanol. For the purpose of high yield ethanol production by the present organisms, metabolic flux is channeled to the oxidoreductase enzymatic pathways by genetically modifying T. saccharolyticum to eliminate competing pathways.

The thermophilic bacterium, T. saccharolyticum, is used by way of example to illustrate how hydrogenase activities in an organism may be manipulated to increase ethanol production. The methods and materials disclosed herein may however apply to members of the Thermoanaerobacter and Thermoanaerobacterium genera, as well as other microorganisms. Members of the Thermoanaerobacter and Thermoanaerobacterium genera may include, for example, Theunoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and/or progeny thereof. Both the carbon-centered and the electron-centered approaches for maximizing ethanol production from biomass may be applicable in metabolic engineering of other microorganisms, such as yeast or fungi.

Major groups of bacteria include eubacteria and archaebacteria. Thermophilic eubacteria include: phototropic bacteria, such as cyanobacteria, purple bacteria and green bacteria; Gram-positive bacteria, such as Bacillus, Clostridium, lactic acid bacteria and Actinomyces; and other eubacteria, such as Thiobacillus, Spirochete, Desulfotomaculum, Gram-negative aerobes, Gram-negative anaerobes and Thermotoga. Within archaebacteria are considered Methanogens, extreme thermophiles (an art-recognized term) and Thermoplasma. In certain embodiments, the present instrumentalities relate to Gram-negative organotrophic thermophiles of the genus Thermus; Gram-positive eubacteria, such as Clostridium, which comprise both rods and cocci; eubacteria, such as Thermosipho and Thermotoga; archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus and Methanopyrus. Some examples of thermophilic or mesophilic organisms (including bacteria, prokaryotic microorganisms and fungi), which may be suitable for use with the disclosed instrumentalities include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Anaerocellum sp., Thermoanaerobacterium thermosaccharolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, variants thereof, and/or progeny thereof.

In certain embodiments, thermophilic bacteria for use with the disclosed instrumentalities may be selected from the group consisting of Fervidobacterium gondwanense, Clostridium thermolacticum, Moorella sp. and Rhodothermus marinus.

In certain embodiments, the disclosed instrumentalities relate to microorganisms of the genera Geobacillus, Saccharococcus, Paenibacillus, Bacillus and Anoxybacillus, including but not limited to species selected from the group consisting of: Geobacillus thermoglucosidasius, Geobacillus stearotheimophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and/or progeny thereof.

In certain embodiments, the disclosed instrumentalities relate to mesophilic bacteria selected from the group consisting of Saccharophagus degradans; Flavobacterium johnsoniae; Fibrobacter succinogenes; Clostridium hungatei; Clostridium phytofeimentans; Clostridium cellulolyticum; Clostridium aldrichii; Clostridium termitididis; Acetivibrio cellulolyticus; Acetivibrio ethanolgignens; Acetivibrio multivorans; Bacteroides cellulosolvens; and Alkalibacter saccharofomentans, variants thereof, and/or progeny thereof.

In certain preferred embodiments, the disclosed instrumentalities relate to organisms having a ferredoxin-linked hydrogenase (EC subclass 1.12.7.2), including but not limited to organisms selected from the groups of eubacteria and achaebacteria, phototropic bacteria (such as cyanobacteria, purple bacteria and green bacteria), Gram-positive bacteria and lactic acid bacteria and Gram-negative anaerobes, as well as organisms selected from the genera including, but not limited to: Bacillus, Clostridium, Thermotoga, Pyrococcus and Saccharococcus. Such organisms include those selected from the group consisting of: Thermotoga maritima, Clostridium acetobutylicum, Clostridium pasteurianum, Clostridium beijerinckii, Clostridium thermosulfurogenes, Clostridium cellulolyticum, Clostridium thermocellum, Clostridium thermohydrosulfuricum, Clostridium thermosaccharolyticum, Clostridium tartarivorum, Clostridium thermocellulaseum, Thermoanaerobacterium thermosaccharolyticum, Thermoanaerobacterium saccharolyticum, Thermobacteroides acetoethylicus, Thermoanaerobium brockii, Pyrococcus furiosus, Bacillus coagulans, Clostridium thermolacticum, Clostridium hungatei, Clostridium phytofermentans, Clostridium cellulolyticum, Clostridium aldrichii, Clostridium termitididis, Acetivibrio cellulolyticus, Acetivibrio ethanolgignens, Acetivibrio multivorans, Bacteroides cellulosolvens, Alkalibacter saccharofomentans, variants thereof, and/or progeny thereof.

Two hydrogenases, hyd and hydtr, have been identified in T. saccharolyticum. The hyd and hydtr hydrogenases are each composed of four subunits, A-D, which are encoded by four different genes, respectively. The hydA gene encodes subunit A of the hyd hydrogenase, while the hydtrA gene encodes subunit A of the hydtr hydrogenase. The identity and function of these two hydrogenases have been confirmed based on enzymatic activity assays and comparative analysis of genomic sequences. Inactivation of these two hydrogenases, alone or in combination, by site-directed gene knockout is disclosed herein. The resulting mutant strains no longer possess the hydrogenase activity specific for a native strain.

In an aspect, an isolated polynucleotide comprises: (a) the nucleotide sequence of hydA (SEQ ID NO: 1) or fragment thereof; (b) the nucleotide sequence of hydB (SEQ ID NO: 2) or fragment thereof; (c) the nucleotide sequence of hydC (SEQ ID NO: 3) or fragment thereof; (d) the nucleotide sequence of hydD (SEQ ID NO: 4) or fragment thereof; (e) the nucleotide sequence of hydtrA (SEQ ID NO: 8) or fragment thereof; (f) the nucleotide sequence of hydtrB (SEQ ID NO: 5) or fragment thereof; (g) the nucleotide sequence of hydtrC (SEQ ID NO: 6) or fragment thereof; (h) the nucleotide sequence of hydtrD (SEQ ID NO: 7) or fragment thereof; or (i) a nucleotide sequence encoding a hydrogenase or a subunit thereof with substantially similar activity as the hydrogenase or subunit encoded by one of the sequences selected from (a)-(h), said nucleotide sequence also having at least about 90%, 95%, 98%, or 99% sequence identity with the corresponding sequence selected from (a)-(h). In another aspect, a vector comprising at least one polynucleotide sequence selected from (a)-(i) is disclosed.

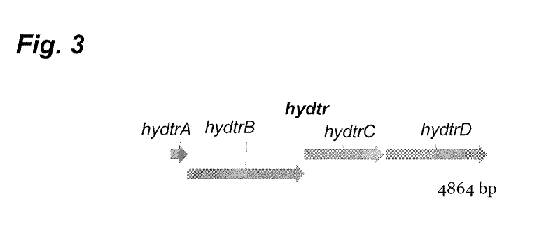

The four subunits of the hyd hydrogenase encoded by hydA, hydB, hydC, and hydD, may be referred to as hydA protein (or subunit) (SEQ ID NO: 9), hydB protein (or subunit) (SEQ ID NO: 10), hydC protein (or subunit) (SEQ ID NO: 11), and hydD protein (or subunit) (SEQ ID NO: 12), respectively. A genetic map of the hydA-hydD genes is shown in FIG. 2. Similarly, the four subunits of the hydtr hydrogenase encoded by hydtrA, hydtrB, hydtrC, and hydtrD, respectively, may be referred to as hydtrA protein (or subunit) (SEQ ID NO: 16), hydtrB protein (or subunit) (SEQ ID NO: 13), hydtrC protein (or subunit) (SEQ ID NO: 14), and hydtrD protein (or subunit) (SEQ ID NO: 15), respectively. A genetic map of the hydtrA-hydtrD genes is shown in FIG. 3. It is conceivable that a protein with substantial sequence similarity to one of the polypeptides of SEQ ID NOS: 9-16 may have substantially similar functionality or activity as the corresponding hyd or hydtr hydrogenase subunit. For purposes of this disclosure, other proteins having hydrogenase activity and sharing at least about 70% sequence identity with one of the proteins selected from SEQ ID NOS: 9-16 may be used to function as a hydrogenase or its subunit in place of the corresponding hyd or hydtr subunit. More preferably, such other proteins share at least 90%, 95%, 98% or 99% sequence identity with one of the proteins selected from SEQ ID NOS: 9-16.

In an aspect, an organism that contains at least one hydrogenase gene may be genetically altered by eliminating or downregulating expression of the at least one hydrogenase gene. Expression of the hydrogenase gene may be disrupted, for example, by deletion, insertion, point mutation(s), or by otherwise rendering expression of a functional hydrogenase encoded by the gene unfavorable. Both the coding and non-coding regions of a hydrogenase gene may be altered to affect hydrogenase activity.

In another aspect, the organism with decreased hydrogenase activity may contain additional mutations which eliminate or reduce the ability of the organism to produce lactic acid and/or acetic acid. For example, lactate dehydrogenase (ldh), the gene that confers the ability to produce lactic acid, and acetate kinase (ack) and/or phosphotransacetylase (pta), the genes that confer the ability to produce acetic acid, may be targeted for gene disruption as described in PCT/US07/67941, which is incorporated by reference herein.

Inactivation of hydA in T. saccharolyticum results in no measurable changes in the production of acetic acid, hydrogen, and ethanol by the mutant strain when compared to the parental strain. One explanation of this result is that the hydA hydrogenase may catalyze the transfer of electrons from NAD(P)H to hydrogen, which may not be a significant metabolic pathway in pure culture or under process conditions used for ethanol production. Under the conditions described above, hydrogen production from NAD(P)H may be thermodynamically unfavorable, and electrons may be transferred from the electron carrier ferredoxin to hydrogen, which may be thermodynamically more favorable under these conditions. See, Thauer, R. K., K. Jungermann, and K. Decker (1977) Energy conservation in chemotrophic anaerobic bacteria. Microbiol. Mol. Biol. Rev. 41: 100-180.

While inactivation of hydA resulted in a bacterial strain with no measurable change in acetic acid, hydrogen, and ethanol production compared to the non-engineered strain, inactivation (also known as “knockout”) of hydtrA resulted in a bacterial strain with significant reduction in hydrogen and acetic acid production compared to the non-engineered strain. As expected, the hydtr knockout strain also showed increased production of lactic acid and ethanol. It is shown here that inactivation of hydtrA decreases hydrogen production by over 90% and acetic acid production by more than 80% compared to the non-engineered strain. In addition, ethanol production was increased by 20% and lactic acid production was increased by 150% compared to the non-engineered strain.

An organism may be able to express more than one hydrogenase. Under normal conditions, only the primary hydrogenases are expressed and functional. The expression of other hydrogenases (secondary hydrogenases) may be induced only after certain primary functional hydrogenases have been inactivated. Under certain conditions, the secondary hydrogenases may be able to completely take over the function of the primary hydrogenases, and no phenotypic changes may be observed. It may thus be desirable to identify all such functionally redundant hydrogenases in an organism and inactivate all of them so that the electron flow may be effectively directed to a particular intermediate or end product in a metabolic pathway.

In an aspect, an organism may be generated in which all hydrogenase activities leading to synthesis of hydrogen are disrupted in order to maximize ethanol production. For instance, both the hyd and hydtr hydrogenases may be inactivated to remove the residual hydrogen production observed in the hydtr single KO strain. Such elimination of hydrogenase activity may be achieved using two site-directed DNA homologous recombination events to knockout both hyd and hydtr.

The present disclosure shows the genomic organization of genes encoding hydrogenases in the thermophilic bacterium T. saccharolyticum. Two hydrogenase systems have been identified in T. saccharolyticum based on enzymatic activity assays and analysis of the genomic sequence. A subunit of hydA in T. saccharolyticum shares significant sequence identity with the hydA subunit of an Fe-only hydrogenase in Clostridia and the NAD(H) dependent Fe-only hydrogenase in Thermoanaerobacter tengcongensis. (Soboh, B., D. Linder, and R. Hedderich (2004) A multisubunit membrane-bound [NiFe] hydrogenase and an NADH-dependent Fe-only hydrogenase in the fermenting bacterium Thermoanaerobacter tengcongensis. Microbiology 150: 2451-2463.) The hydA gene encodes a polypeptide subunit of a multi-subunit hydrogenase in Thermoanaerobacter tengcongensis. The hydtrA-containing hydrogenase likely plays a role in catalyzing the transfer of electrons from ferredoxin to hydrogen. The genomic organization of the genes encoding the subunits of hyd and hydtr hydrogenase operons in T. saccharolyticum are shown in FIGS. 2 and 3.

In another aspect, it may be desirable to combine the “carbon-centered” approach with the “electron-centered” approach in order to direct the flow of carbon and electrons to a specific intermediate or end product. To this end, additional genes encoding proteins other than a hydrogenase may be disturbed in a hydrogenase knockout strain. For example, a hydtrA and L-ldh double knockout strain designated HLK1 is described herein. Results from the HLK1 strain suggest that an “electron-centered” approach may be used to create a metabolically engineered microorganism that produces ethanol as a primary fermentation product. In comparison to the L-ldh single knockout strain reported by Desai et al. (2004), HLK1 produces 77% less acetic acid and 36% more ethanol in batch fermentation with 5 grams per liter cellobiose and 5 grams per liter yeast extract.

The hydrogenase knockout strains (i.e., hyd and/or hydtr knockouts) and other knockout strains wherein one or more of ldh, ack and pta is knocked out in combination with one or more of the hydrogenase genes, may contribute significant cost savings to the conversion of biomass to ethanol due to their growth conditions, which are substantially optimal for cellulase activity in SSF and SSCF processes. For example, optimal cellulase activity parameters include a pH between 4-5 and temperature between 40-50° C., which are substantially similar to the optimal growth conditions of thermophilic bacteria. By way of comparison, the optimal growth temperature for T. saccharolyticum is about 50-60° C. (Esterbauer, H., W. Steiner, I. Labudova, A. Hermann, and M. Hayn. (1991) Production of Trichoderma Cellulase in Laboratory and Pilot Scale. Bioresource Technology 36: 51-65.) Thus, if the reaction is carried out within the temperature range of 40-60° C., the biocatalysts and cellulases may both achieve their maximal activities. One benefit of this overlap in optimal temperature is that the amount of cellulase required for producing the same amount of ethanol may be lowered by as much as two-thirds resulting in a significant cost reduction. See, e.g., Mabee, W. E. and J. N. Saddler (2005) Progress in Enzymatic Hydrolysis of Lignocellulosics. In Anonymous. Additionally, it is unnecessary to adjust the pH of the fermentation broth when knockout organisms, which lack the ability to produce organic acids, are used. These knockout organisms may also be suitable for a consolidated bioprocessing co-culture fermentation where cellulose may be degraded by a cellulolytic organism such as C. thermocellum and these knockout organisms may convert pentoses to ethanol. C. thermocellum is capable of rapidly degrading cellulose, but it cannot ferment pentose sugars, which, in the form of xylan and other polysaccharides, may account for up to 30% of total carbohydrates in a typical saccharified biomass. By contrast, T. saccharolyticum is capable of fermenting and utilizing pentose sugars. A process utilizing both C. thermocellum and a knockout of T. saccharolyticum may therefore be an efficient way to improve cellulosic ethanol production, and reduce process costs. See Lynd, L. R., W. H. van Zyl, J. E. McBride, and M. Laser (2005) Consolidated bioprocessing of cellulosic biomass: an update. Curr. Opin. Biotechnol. 16: 577-583.

Operating either an SSF, SSCF or CBP process at thermophilic temperatures offers several important benefits over conventional mesophilic fermentation temperatures of 30-37° C. In particular, enzyme concentrations necessary to achieve a given amount of conversion may be reduced due to higher enzyme activity at thermophilic temperatures. As a result, costs for a process step dedicated to cellulase production are substantially reduced for thermophilic SSF and SSCF (e.g., 2-fold or more), and are eliminated for CBP. Costs associated with fermentor cooling and heat exchange before and after fermentation are also expected to be reduced for thermophilic SSF, SSCF and CBP. Finally, processes featuring thermophilic biocatalysts may be less susceptible to microbial contamination as compared to processes featuring conventional mesophilic biocatalysts.

In an aspect, a method for producing ethanol includes providing within a reaction vessel, a reaction mixture comprising lignocellulosic substrate, a cellulolytic material and a fermentation agent. The fermentation agent comprises an organism that has been transformed to eliminate expression of at least one gene encoding a hydrogenase. The reaction mixture is reacted under suitable conditions for a period of time sufficient to allow saccharification and fermentation of the lignocellulosic substrate. Appropriate substrates for the production of ethanol include, for example, one or more of glucose, xylose, cellobiose, sucrose, xylan, starch, cellulose, pectin and combinations thereof. These substrates may, in some aspects, be produced during an SSF, SSCF or CBP process to achieve efficient conversion of biomass to ethanol.

It will be appreciated that carbohydrate-rich biomass material that is saccharified to produce one or more of glucose, xylose, mannose, arabinose, galactose, fructose, cellobiose, sucrose, maltose, xylan, mannan, starch cellulose and pectin may be utilized by the disclosed organisms. In various embodiments, the biomass may be lignocellulosic biomass that comprises wood, corn stover, sawdust, bark, leaves, agricultural and forestry residues, grasses such as switchgrass, ruminant digestion products, municipal wastes, paper mill effluent, newspaper, cardboard, or combinations thereof.

### Deposit of HLK1

HLK1 has been deposited with the American Type Culture Collection, Manassas, Va. 20110-2209. The deposit was made on Jan. 17, 2008 and received Patent Deposit Designation Number PTA-8897. This deposit was made in compliance with the Budapest Treaty requirements that the duration of the deposit should be for thirty (30) years from the date of deposit or for five (5) years after the last request for the deposit at the depository or for the enforceable life of a U.S. patent that matures from this application, whichever is longer. HLK1 will be replenished should it become non-viable at the depository.

### Example 1

### Identification and Sequencing of Target Hydrogenase Genes in Thermoanaerobacterium saccharolyticum

### Materials and Methods

Thermoanaerobacterium saccharolyticum strain JW/SL-YS485 (DSM 8691) is a thermophilic, anaerobic bacteria isolated from the West Thumb Basin in Yellowstone National Park, Wyoming. (Lui, S. Y., F. C. Gherardini, M. Matuschek, H. Bahl, J. Wiegel (1996) Cloning, sequencing, and expression of the gene encoding a large S-layer-associated endoxylanase from Thermoanaerobacterium sp strain JW/SL-YS485 in Escherichia coli. J. Bacteriol. 178: 1539-1547; Mai, V., J. Wiegel (2000) Advances in development of a genetic system for Thermoanaerobacterium spp: Expression of genes encoding hydrolytic enzymes, development of a second shuttle vector, and integration of genes into the chromosome. Appl. Environ. Microbiol. 66: 4817-4821, 2000.) It grows in a temperature range of 30-66° C. and a pH range of 3.85-6.5. It consumes a variety of biomass derived substrates including the monosaccharides glucose and xylose, the disaccharides cellobiose and sucrose, and the polysaccharides xylan and starch. The organism produces ethanol as well as the organic acids lactic acid and acetic acid as primary fermentation products.

### Cloning and Sequencing

Genes encoding the hyd subunits were identified and sequenced using standard techniques, as reported previously by Desai et al. (2004). Degenerate primers were designed using the CODE-HOP algorithm (Rose, T., E. Schultz, J. Henikoff, S. Pietrokovski, C. McCallum, S. Henikoff (1 Apr. 1998) Consensus-degenerate hybrid oligonucleotide primers for amplification of distantly-related sequences. Nucleic Acids Research, 26(7): 1628-1635) and PCR reactions were performed to obtain the DNA sequence between conserved regions. The gene fragments outside of the conserved regions were sequenced directly from genomic DNA using ThermoFidelase (Fidelity Systems, Gaithersburg, Md.) enzyme with BigDye Terminator kit v3.1 (ABI, Foster City Calif.).

The genes encoding the hydtr subunits were identified based on homology to known hydrogenases from the genomic sequence of T. saccharolyticum, which had been sequenced by the method of shotgun sequencing (Agencourt, Beverly, Mass.).

### Construction of Vectors

A gene inactivation “knockout” vector, pHydKO, targeting the hydA gene was created using standard cloning methods. (Sambrook, J. and D. W. Russell. (2001) Molecular cloning: a laboratory manual. Cold Spring Harbor Laboratory.) This knockout vector utilized the method of homologous recombination to integrate into the chromosome upstream and downstream of the hydA gene, resulting in replacement of the hydA gene with the erythromycin antibiotic resistance gene. pHydKO was created with DNA fragments from pBLUESCRIPT II SK (+) (Stratagene, Cedar Creek, Tex.) cut by the restriction enzymes Xhol and Sacl (New England Biolabs, Ipswich, Mass.); DNA homologous to the 5′ upstream region of hydA amplified from T. saccharolyticum genomic DNA via PCR with primer pair 1 and 2, and subsequently digested with the restriction enzymes Xhol and Xbal; DNA homologous to the 5′ downstream region of hydA amplified from T. saccharolyticum genomic DNA via PCR with the primer pair 3 and 4, and subsequently digested with the restriction enzymes Mfel and Sacl; and DNA containing the hybrid kanamycin promoter-erythromycin resistance gene described by Klapatch et al. from plasmid pSGD8-erm digested by Xbal and EcoRl. (Klapatch, T. R., M. L. Guerinot, and L. R. Lynd. (1996) Electrotransformation of Clostridium thermosaccharolyticum. J. Ind. Microbiol. 16: 342-347.) These four DNA fragments were purified and ligated with T4 DNA ligase (New England Biolabs), purified again and transformed into competent E. coli DH5α (Invitrogen, Carlsbad, Calif.) and selected for with ampicillin at 100 μg/mL and erythromycin at 200 μg/mL. A single colony derived plasmid with the correct construction was retained as pHydKO.

A gene inactivation “knockout” vector, pHydtrKO, targeting the hydtrA gene was created using standard cloning methods (Sambrook, et al. (2001)). This knockout vector utilized the method of homologous recombination to integrate into the chromosome upstream and downstream of the hydtrA gene, resulting in replacement of the hydtrA gene with the kanamycin antibiotic resistance gene. pHydtrKO was created with DNA fragments from pBLUESCRIPT II SK (+) cut by the restriction enzymes Xhol and Eagl; DNA homologous to the 5′ upstream region of hydtrA amplified from T. saccharolyticum genomic DNA via PCR with the primer pair 5 and 6, and subsequently digested with the restriction enzymes Xhol and Pstl; DNA homologous to the 5′ downstream region of hydtrA amplified from T. saccharolyticum genomic DNA Via PCR with the primer pair 7 and 8, and subsequently digested with the restriction enzymes EcoRl and Eagl; and DNA containing the kanamycin resistance gene from plasmid pIKMl described by Mai et al. digested by Pstl and EcoRl. (Mai, V., Lorenz, W. W. and J. Wiegel. (1997) Transformation of Thermoanaerobacterium sp. strain JW/SL-YS485 with plasmid pIKMl conferring kanamycin resistance. FEMS Microbiol. Lett. 148: 163-167.) These four DNA fragments were purified and ligated with T4 DNA ligase, purified again and transformed into competent E. coli DH5α and selected for with ampicillin at 100 μg/mL and kanamycin at 50 μg/mL. A single colony derived plasmid with the correct construction was retained as pHydtrKO.

A gene inactivation “knockout” vector, pSGD8-Erm, targeting the L-ldh gene was created using standard cloning methods (Sambrook, et al. (2001)) based on the plasmid pSGD8 of Desai, et al. (2002). In place of the aph kanamycin antibiotic marker, a fusion gene based on the aph promoter from the plasmid pIKMl and the adenine methylase gene conferring erythromycin resistance from the plasmid pCTCl were used for selection. PCR gene fragments were created using pfu polymerase (Statagene) and the primer pair 9 and 10 for the aph promoter and primer pair 11 and 12 for the adenine methylase open reading frame. Fragments were digested with XbaI/BamHl (aph fragment) and BamHI/EcoRI (adenine methylase) and ligated into the multiple cloning site of pIKMl. This fusion gene was then excised with BseRI/EcoRI and ligated into similarly digested pSGD8.

The sequences of the primer pairs are as follows:

Primer 1
(SEQ ID. No. 17)
5′ TTACTCGAGAAACTGGTGGAACATCTGGTGGAT3′
Primer 2
(SEQ ID. No. 18)
5′ AAGTCTAGATAAATCGCTCCGACAGGACATGCT3′
Primer 3
(SEQ ID. No. 19)
5′ CTACAATTGGACTTGCCTATCAGAAAGTCTCACA3′
Primer 4
(SEQ ID. No. 20)
5′ ATAGAGCTCTCATGGGAGAACCAGATGCAAGTA3′
Primer 5
(SEQ ID. No. 21)
5′ ATATCTCGAGCTGTAATTGTCCTTGATGACG3′
Primer 6
(SEQ ID. No. 22)
5′ ATATCTGCAGCAGGATATGATGGAGCTACAGTG3′
Primer 7
(SEQ ID. No. 23)
5′ ATATGAATTCCATATATGAGAGGGAGGGCTGA3′
Primer 8
(SEQ ID. No. 24)
5′ ATATCGGCCGAGTCGTTTCTCCTAACAAG3′
Primer 9
(SEQ ID. No. 25)
5′ TGGATCCGCCATTTATTATTTCCTTCCTCTTTTC3′
Primer 10
(SEQ ID. No. 26)
5′ TTCTAGATGGCTGCAGGTCGATAAACC3′
Primer 11
(SEQ ID. No. 27)
5′ GCGGATCCCATGAACAAAAATATAAAATATTCTC3′
Primer 12
(SEQ ID. No. 28)
5′ GCGAATTCCCTTTAGTAACGTGTAACTTTCC3′

Transformation of T. saccharolyticum

Transformation of T. saccharolyticum was performed with the following two methods. The first was as previously described by Mai, et al. (1997). The second method had several modifications following cell harvest and was based on the method developed for Clostridium thermocellum. (Tyurin, M. V., S. G. Desai, L. R. Lynd, (2004) Electrotransformation of Clostridium thermocellum. Appl. Environ. Microbiol. 70(2): 883-890.) Briefly, cells were grown overnight using pre-reduced medium DSMZ 122 in sterile disposable culture tubes inside an anaerobic chamber in an incubator maintained at 55° C. Thereafter, cells were sub-cultured with 4 μg/ml isonicotonic acid hydrazide (isoniacin), a cell wall weakening agent (Hermans, J., J. G. Boschloo, J. A. M. de Bont (1990) Transformation of M. aurum by electroporation: The use of glycine, lysozyme and isonicotinic acid hydrazide in enhancing transformation efficiency. FEMS Microbiol. Lett. 72: 221-224) added to the medium after the initial lag phase. Exponential phase cells were harvested and washed with pre-reduced cold sterile 200 mM cellobiose solution, and resuspended in the same solution and kept on ice. Cells were kept cold (approximately 4° C.) during this process.

Samples composed of 90 μl of the cell suspension and 2 to 6 μl of the knockout or control vector (1 to 3 μg) added just before pulse application, were placed into sterile 2 ml polypropylene microcentrifuge disposable tubes that served as electrotransformation cuvettes. A square-wave with pulse length set at 10 ms was applied using a custom-built pulse generator/titanium electrode system. A voltage threshold corresponding to the formation of electropores in a cell sample was evaluated as a non-linear current change when pulse voltage was linearly increased in 200V increments. A particular voltage that provided the best ratio of transformation yield versus cell viability rate at a given DNA concentration was used. The voltage used in this experiment was 25 kV/cm. Pulsed cells were initially diluted with 500 μl DSM 122 medium, held on ice for 10 minutes and then recovered at 55° C. for 4-6 hrs. Following recovery, cells transformed with the control vector were mixed with medium containing 1% agar and either kanamycin at 200 μg/ml or erythromycin at 10 μg/ml and poured onto petri plates with media at pH 6.7 for kanamycin selection or pH 6.1 for erythromycin selection and incubated in anaerobic jars for 4 days at 52° C. Other media that can support growth of T. saccharolyticum may also be used. The transformed cell lines may be used without further manipulation. Subsequent transformations may be performed in a similar fashion if desired to obtain an organism with additional genes inactivated. The second transformation may be carried out as described above with the primary transformant substituted for the non-transformed cell suspension.

T. saccharolyticum strains with either the hydtr or hydA gene inactivated were created by transformation of wild-type T. saccharolyticum with appropriate constructs as described above. L-ldh KO strain was generated as previously described in Desai et al. (2004). A T. saccharolyticum strain (designated HLK1) with both hydtr and L-ldh inactivated was obtained by transformation of the L-ldh KO strain with the construct described above to inactivate hydtr in a L-ldh KO background. Similarly, another double-knockout strain was generated where both L-ldh and hydA were inactivated.

### Verification of Mutant Strains

Site-directed recombination regions were identified by PCR from genomic DNA extracted from various single or double knockout strains using Taq polymerase (New England Biolabs) and primers outside and inside the regions of homologous overlap between the genome and the constructs. PCR products of the expected size resulting from one internal and one external primer spanning the homology overlap in both directions were taken as confirmation for a double site integration. The L-ldh, hydtr and/or hydA loci deletions all involved a double integration, a more genetically stable embodiment of the gene knockout process.

### Example 2

### Hydrogenase Gene Expression Levels and Enzymatic Activities in Thermoanaerobacterium saccharolyticum

RT-PCR was used to measure mRNA levels of hydrogenase genes in T. saccharolyticum (Table 1). The level of 16S rRNA was used to normalize the data.

TABLE 1
Transcript Levels of Certain Hydrogenase
Genes in T. saccharolyticum

| Gene Name | Transcript Levels Relative to 16S rRNA |
|---|---|
| hydA | 16 |
| hydtrB | 0.82 |
| hydtrD | 0.6 |

The level and co-factor specificity of hydrogenase activities were analyzed. Briefly, whole cell extract (WCE) was prepared under anaerobic conditions with a French pressure cell. The cells were treated with DNAseI for 30 min at 37° C. and centrifuged at 5000×g for 5 min to remove unbroken cells. Enzymatic assays were performed on the cell free extract and results are shown in Table 2. Hydrogenase activity was observed at 60° C. in the direction of hydrogen formation with the broad range electron donor methyl viologen, and hydrogenase activity specific to NADH, NADPH, and ferredoxin-linked metronidazole reduction were also observed. The following assay conditions were used:

Hydrogenase (EC 1.12) Methyl viologen:H₂ (hydrogen production)—100 mM EPPS (pH 8.0), 1 mM methyl viologen, and 5 mM sodium dithionite. (F. Bryant and M. Adams. (1989) Characterization of hydrogenase from the hyperthermophilic archaebacterium, Pyrococcus furiosus, J. Biol. Chem. 264: 5070-5079.)

Hydrogenase (EC 1.12.7.2) H₂:ferredoxin:metronidazole (hydrogen consumption)—100 mM EPPS (pH 8.0), 1 atm hydrogen, 7.5 ug/mL ferredoxin (C. pasteurianum) and 0.2 mM metronidazole. (Soboh, et al. (2004))

Hydrogenase (EC 1.12.1.2 and EC 1.12.1.3) H₂:NAD(P)H—(hydrogen consumption)—100 mM EPPS (pH 8.0), 1 atm hydrogen, 1.5 mM NAD⁺ or NADP⁺. (Soboh, et al. (2004))

TABLE 2
Specific Activities of Hydrogenase Activities

| Specific activity | | |
|---|---|---|
| (μmol min⁻¹ mg⁻¹) | S.D. | Assay Conditions* |
| 0.22 | 0.12 | MV, H₂ formation, 60° C. |
| | | metronidazole, H₂ uptake, |
| 2.61 | 0.02 | 60° C. |
| 0.041 | 0.018 | NAD⁺, H₂ uptake, 60° C. |
| 0.033 | 0.001 | NADP⁺, H₂ uptake, 60° C. |

*Assay conditions abbreviations. NADH: assayed in direction of NADH oxidation, NADPH: assayed in direction of NADPH oxidation, MV: assayed with methyl viologen, metronidazole: assayed with metronidazole linked to ferredoxin reduction.

Cell free extracts of strains with hyd and hydtr deletions in all four subunits were also assayed for methyl viologen hydrogenase activity (Table 3). Glucose-6-phosphate dehydrogenase was utilized as a control under similar conditions with an assay mixture of 50 mM Tris-HCl, pH 7.6, D-glucose 6-phosphate, NADP, and 30-40 μg cell extract. The hyd knockout strain showed a more than 50% decrease in methyl viologen hydrogenase activity relative to the wildtype, but with nearly identical hydrogen yields. This behavior implies that the natural substrate of the hyd enzyme is NAD(P)H. The hydtr knockout strain had a methyl viologen hydrogenase activity slightly lower than the wildtype, while cell extract from a hydtr, hydA double-knockout strain showed no detectable activity, suggesting that these two enzymes are responsible for methyl viologen hydrogenase activity. (Noltmann, E. A., C. J. Gubler, and S. A. Kuby (1961) Glucose 6-Phosphate Dehydrogenase (Zwischenferment). I. Isolation of the Crystalline Enzyme from Yeast. J. Biol. Chem. 236: 1225-1230.)

TABLE 3
Hydrogenase Enzymatic Activities

| | Methyl Viologen | G6PDH Specific |
|---|---|---|
| | Hydrogenase | Activity |
| | Specific Activity | (μmol/min · mg protein) |
| | (μmol/min · mg protein) | (control assay) |
| Wildtype | 1.70 ± 0.22 | 0.018 ± 0.007 |
| hyd knockout | 0.61 ± 0.12 | 0.022 ± 0.004 |
| hydtr knockout | 1.55 ± 0.29 | 0.022 ± 0.008 |
| hydtr, hydA | −0.02 ± 0.00 | 0.020 ± 0.013 |
| double-knockout | | |

### Example 3

### Fermentation Profiles of Wildtype T. saccharolyticum and hydtr or hydA Single Knockout Strains

Wildtype and mutant T. saccharolyticum strains were grown in partially defined MTC media containing 2.5 g/L Yeast Extract and 5 g/L cellobiose at 56° C. (Zhang, Y., L. R. Lynd (2003) Quantification of cell and cellulase mass concentrations during anaerobic cellulose fermentation: Development of an enzyme-linked immunosorbent assay-based method with application to Clostridium thermocellum batch cultures. Anal. Chem. 75: 219-222). After 25 hours, the final concentrations of cellobiose, acetic acid, lactic acid, ethanol and hydrogen were analyzed by HPLC on an Aminex HPX-87H column (BioRad Laboratories, Hercules, Calif.) at 55° C. The mobile phase was 5 mM sulfuric acid at a flow rate of 0.7 ml/min. Detection was via refractive index using a Waters 410 refractometer (Milford, Mass.). The minimum detection level for acetate was 1.0 mM. Hydrogen was analyzed by gas chromatography on a silica gel column with nitrogen as the carrier gas using a TCD detector (SRI Instruments, Torrance, Calif.).

Carbon balances were determined according to the following equations, with accounting of carbon dioxide through the stoichiometry relationship of its production to acetic acid and ethanol. The carbon contained in the cell mass was estimated by the general formula for cell composition, CH₂N_{0.25}O_{0.5}.

$${C}_{t} = \frac{144}{342} C B + \frac{72}{180} G + \frac{36}{90} L + \frac{35}{60} A + \frac{36}{46} E + \frac{12}{25.5} C D W$$

Cₜ=total carbon, CB=cellobiose, G=glucose, L=lactic acid, E=ethanol, CDW=cell dry weight. All units are expressed in grams per liter (g/L).

$${C}_{R} = \frac{{C}_{tf}}{{C}_{to}} \times 100 \%$$

C_{R}=carbon recover, Cₜₒ=total carbon at the initial time, C_{tf}=total carbon at the final time.

As shown in Table 4, inactivation of hydtr decreased hydrogen production by over 90%, and acetic acid production by more than 80%. Ethanol production increased by about 20% and lactic acid production increased by 150% compared to the non-engineered wildtype strain. By contrast, inactivation of the hydA gene resulted in a bacterial strain with no measurable change in the production of acetic acid, hydrogen, or ethanol compared to the wildtype strain (data not shown).

TABLE 4
Fermentation profiles of wildtype and hydtr KO strains

| | Cello- | | Lactic | | Acetic | | | | H₂ | | Carbon | |
|---|---|---|---|---|---|---|---|---|---|---|---|---|
| | biose | | Acid | | Acid | | Ethanol | | (mM) | | Recovery | |
| | Conc. | SD | Conc. | SD | Conc. | SD | Conc. | SD | Conc. | SD | (%) | |
| Media | 5.14 | 0.01 | 0.00 | 0.00 | 0.00 | 0.00 | 0.00 | 0.00 | 0.00 | 0.00 | 100 | 1 |
| Only | | | | | | | | | | | | |
| Wildtype | 0.20 | 0.08 | 0.79 | 0.08 | 1.20 | 0.01 | 1.69 | 0.01 | 27.47 | 0.67 | 83 | 2 |
| hydtrKO | 0.48 | 0.00 | 1.98 | 0.07 | 0.09 | 0.01 | 2.03 | 0.06 | 0.85 | 0.11 | 96 | 1 |

Concentrations are in grams per liter with the exception of hydrogen in mM.
Standard deviations (SD) are based upon three replicate fermentations.

### Example 4

### Fermentation Profiles of Lactic Acid Knockout (ldh KO), hydtrA-ldh Double Knockout (HLK1), and hydA-ldh Double Knockout Strains

ldh KO, hydtrA-ldh double KO (HLK1), and hydA-ldh double KO were grown and the final concentrations of cellobiose, acetic acid, lactic acid, and ethanol were measured at the end of the incubation period as described in Example 3.

As shown in Table 5 below, HLK1 produced 77% less acetic acid and 36% more ethanol when compared to the L-ldh single knockout strain. By contrast, hydA-ldh double KO showed a similar fermentation profile as the ldh KO strain, consistent with results from the hydA single KO strain. HLK1 produced ethanol at a yield of 0.45 grams ethanol per gram of carbohydrate consumed, which is comparable to strain ALK2, described in PCT/US07/67941.

TABLE 5
Fermentation profiles of the lactic acid knockout (ldhKO),
hydtrA-ldh knockout (HLK1), and hydA-ldh knockout

| | | Lactic | Acetic | | Carbon |
|---|---|---|---|---|---|
| | Cellobiose | Acid | Acid | Ethanol | Recovery (%) |
| Media Only | 5.40 | 0.00 | 0.00 | 0.00 | 100 |
| ldhKO | 0.00 | 0.00 | 1.41 | 1.87 | 109 |
| hydtrA-ldhKO | 0.00 | 0.00 | 0.32 | 2.54 | 100 |
| (HLK1) | | | | | |
| hydA-ldhKO | 0.00 | 0.00 | 1.36 | 1.80 | 105 |

Concentrations are in grams per liter.

The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or terminology employed herein is for the purpose of description and not limitation.

All references mentioned in this application are incorporated by reference to the same extent as though fully replicated herein.

What is claimed:

1. An organism capable of fermenting a saccharification product of a carbohydrate-rich biomass substrate, wherein at least one hydrogenase gene endogenous to said organism has been inactivated by genetic engineering.

2. The organism of claim 1, wherein said hydrogenase gene has at least 90% sequence identity with a polynucleotide sequence selected from the group consisting of SEQ ID NOS: 1-8.

3. The organism of claim 1, wherein the organism is a bacterium.

4. The organism of claim 1, wherein the organism is a thermophilic, anaerobic, Gram-positive bacterium.

5. The organism of claim 4, wherein the bacterium is Thermoanaerobacterium saccharolyticum.

6. The organism of claim 1, wherein the at least one hydrogenase gene includes a plurality of genes.

7. The organism of claim 1, wherein at least a second gene encoding a protein other than hydrogenase is inactivated.

8. The organism of claim 7, wherein the second gene encodes a protein that is required by the organism to produce lactic acid as a fermentation product.

9. The organism of claim 8, wherein the second gene is lactate dehydrogenase (ldh).

10. The organism of claim 7, wherein the second gene encodes a protein that is required by the organism to produce acetic acid as a fermentation product.

11. The organism of claim 10, wherein the second gene is selected from the group consisting of acetate kinase (ack) and phosphotransacetylase (pta).

12. A bacterium capable of fermenting a saccharification product of a carbohydrate-rich biomass substrate, wherein ldh and hydtrA genes are inactivated by genetic engineering.

13. A Thermoanaerobacterium saccharolyticum strain deposited under Patent Deposit Designation No. PTA-8897.

14. An isolated polynucleotide comprising a nucleotide sequence having at least 90% sequence identity with a polynucleotide sequence selected from the group consisting of SEQ ID NOS: 1-8.

15. An isolated polynucleotide molecule comprising a polynucleotide sequence selected from the group consisting of SEQ ID NOS: 1-8.

16. A genetically engineered cell expressing a hydrogenase encoded by a gene having at least 90% sequence identity with a nucleotide sequence selected from the group consisting of SEQ ID NOS: 1-8, the expression of said hydrogenase being driven by a heterologous promoter.

17. The genetically engineered cell of claim 16 having been derived from a bacterial cell.

18. The genetically engineered cell of claim 16 having been derived from a yeast cell.

19. A genetic construct comprising a coding sequence having at least 90% sequence identity with a nucleotide sequence selected from the group consisting of SEQ ID NOS: 1-8, said coding sequence being operably linked to a promoter capable of controlling transcription in a bacterial cell.

20. A bacterial cell comprising the genetic construct of claim 19.

21. A method for producing ethanol, said method comprising:
generating an organism with at least one gene encoding a hydrogenase that is inactivated; and
incubating the organism in a medium containing at least one substrate selected from the group consisting of glucose, xylose, mannose, arabinose, galactose, fructose, cellobiose, sucrose, maltose, xylan, mannan, starch, cellulose, pectin and combinations thereof to allow for production of ethanol from the substrate.

22. The method of claim 21, wherein the organism is a member of the Thermoanaerobacterium genus.

23. A method for producing ethanol, said method comprising:
providing within a reaction vessel, a reaction mixture comprising a carbohydrate-rich biomass substrate, a cellulolytic material, and a fermentation agent, the fermentation agent comprising a bacterium that has been genetically modified to inactivate at least one hydrogenase gene endogenous to said bacterium,
wherein the reaction mixture is incubated under suitable conditions for a period of time sufficient to allow saccharification and fermentation of the carbohydrate-rich biomass substrate.

24. The method of claim 23, wherein the cellulolytic material comprises cellulase.

25. The method of claim 23, wherein the cellulolytic material comprises a microorganism capable of hydrolyzing cellulose and hemicellulose into component sugars.

26. The method of claim 23, wherein the suitable conditions comprise a temperature of at least 50° C.

27. The method of claim 23, wherein the bacterium is a member of the Thermoanaerobacterium genus.

28. The method of claim 27, wherein the bacterium is a Thermoanaerobacterium saccharolyticum.

29. The method of claim 23, wherein said hydrogenase gene has at least 90% sequence identity with SEQ ID NO: 8.

30. The method of claim 29, wherein a second gene encoding lactate dehydrogenase is inactivated in the bacterium.

31. An isolated protein molecule having hydrogenase activity, said molecule comprising a polypeptide having an amino acid sequence having at least 90% sequence identity with a polypeptide selected from the group consisting of SEQ ID NOS: 9-16.

32. A bacterium capable of fermenting a saccharification product of a carbohydrate-rich biomass substrate, wherein at least one hydrogenase gene endogenous to said bacterium has been inactivated by genetic engineering.


SEQUENCE LISTING (28 sequences)

SEQ ID NO: 1 - DNA, 1764 bp, Thermoanaerobacterium saccharolyticum
atgaaaggagtgcaaaacatggataaagttcgaataactattgatggaattcctgcagaagtacctgctaactatacagtattgcaagctgcaaaatatgcaaaaattgagattccgacattatgctaccttgaagagataaacgaaataggtgcttgcaggctatgcgttgttgagataaaaggcgttagaaatttacaggcatcttgtgtttatcctgtaagcgacggaatggaaatatacacgaatactcctcgtgtaagagaggcaaggagatctaatttagagcttatactgtctgcacacgacagaagctgccttacatgcgtaagaagcggaaactgtgagttgcaagatttaagtagaaagtctggcatagatgaaataaggtttatgggcgaaaatataaaatatcaaaaagatgagtcgtctccttccatcgtaagagacccaaataaatgcgtattgtgtagaaggtgtgttgctacctgcaacaatgtgcagaatgttttcgccataggcatggttaacagaggatttaagactattgttgcaccttcatttggcagaggtctaaacgaatcaccatgtattagctgcggacagtgtatagaagcatgtcctgtcggagcgatttatgaaaaagaccatacaaagattgtttacgatgcgcttttggatgagaagaaatacgttgtagttcagacagcacctgctgtgagagttgcacttggtgaagagtttggaatgccttatggttcgatagtgacagggaaaatggtatcagctttaaaaaggcttgggtttgacaaagtgtttgacacagactttgctgcagatttaaccataatagaagaaggaaatgaacttttaaagaggcttaacgaaggcggtaagcttcctatgataacatcctgcagccctggatggataaactattgtgaaaggtattatccagaatttatagacaatctttctacttgcaaatcgcctcacatgatgatgggcgcaataataaagagctattttgcggaaaaagaaggaatagatccaaaggatatcttcgttgtatcaattatgccgtgtactgccaagaagtatgagatagacaggcctcaaatgatagtagatggcatgaaagatgtagatgctgttttgacgacgagggagcttgctcgtatgataaaacagtcaggcatagattttgtcaacttgcctgacagcgaatacgacaatccgctgggcgaatcatccggtgctggtgtcatattcggtgctacaggcggtgtcatggaagcggctttaagaactgttgcagatatagttgaaggaaaagatattgagaattttgagtacgaagaagtaagaggattggaaggaataaaagaagcgaagattgacataggcggaaaagaaataaaaatagctgtagcaaatggcacagggaatgctaagaaactcttagacaagataaagaatggcgaggcagagtaccatttcatagaagtcatggggtgccctggcggttgcataatgggcggcggacagccaatacacaatccaaatgaaaaagatttggtgaggaaaagtaggttaaaagccatatatgaagcagataaagacttgcctatcagaaagtctcacaaaaatccaatgataacaaagctgtacgaagaattcttaataagcccattaggagaaaaatctcatcacttgcttcatacaacctatagcaaaaaagatctttatcctatgaatgattaa

SEQ ID NO: 2 - DNA, 1791 bp, Thermoanaerobacterium saccharolyticum
atgttatatagatcacatgttatggtgtgcggtggtactggatgtacatcgtcaaattcagatagaatagcaaaatgctttgaagaagaaattgcaaataaaggtttagacaaagaagttcaggttgtaagaactggatgctttggactttgtgagttgggcccagttgttgtcgtgtatccagaaggcgtgttttacagctgtgtcaaagaagaatatgttccggaaatcgtggaagaacaccttctaaaaggaagagttgttaaaaagtatctttatggagaaagcgtcacagaagaaggaatcaaacctttagaggaaacagcatttttcaagaaacagcagagagttgctttaagaaactgtggtcttataaacccagaggatataaaagaagcaattgcatttgatggctataaagcattggcaaaggtattgactgagatgacgcctgaggaagtcataaatgagattaaaaagtcaggcttaagaggtagaggtggtggtggcttccctacaggtataaagtgggaatttgcttacaaccaaaaagagacgcctaagtacgtcgtttgtaatgctgatgaaggggatcctggtgccttcatggatagaagcgtattggagggagatcctcacagcgttttggaagctatggctatagcaggatatgcaattggtgctaaccatggttatatttatgtaagggctgaatatcctcttgcagtaaagaggcttcaaattgcgatagatcaagcaagagaatacggacttttaggcaaaaatattttcaatacgggatttgactttgatatagagataaggcttggagcaggtgcttttgtctgcggtgaagagactgcacttttaaattctgtcatgggaaaacgcggtgaaccaaggccaaggcctccattccctgctgtaaaaggcgtgtgggaaaaaccaactatcataaacaacgttgaaacttatgcaaatattcctgcgataatattgaatggtgcagaatggttcgcaagtataggcactgaaaaatctaaaggcacaaaggtatttgctcttggcggaaaaatcaacaatactggcttggtagaaatacctatgggtacaaccctgagagagatcatatttgaaataggtggcggaataccaaatggcaagaaattcaaagcagctcaaactggtggaccatctggtggatgcattcctgcggagcatttagatacacctattgactatgattcgcttcttaatattggttccatgatgggttcaggtggacttatcgtaatggacgaagacaactgtatggttgatattgcaaaattcttcttggaatttaccgttgatgaatcatgtggcaaatgctcaccatgtcgcataggtacgagaagaatgttggaactgcttaataagataacatcaggaaagggcgaagaaggagatatcgagaaacttgaaactcttgctaattccataaaggcgtcttctttgtgtggattaggtcaaacagctcctaaccctgttctttccactataaggtattttagagatgaatatgaggcgcacataaaggagaaaaggtgtcctgcaggtgtttgccaggcacttctgaaatttagaattgatccagataaatgtaagggatgcggcatatgtgccaagaattgtcctacaaacgccatatctggaaaagtaaagcagcctcatgtgatagatcaagataaatgtataaaatgtggaacatgtatggataaatgtccgtttgatgctatatacaagaaatag

SEQ ID NO: 3 - DNA, 483 bp, Thermoanaerobacterium saccharolyticum
atgcaggcaatctacgaaaaattcagcgaagaaaatataaataagttaaaaaaagtgatagaccaattgaaagatacagacggttctttgattgctgtcatgaatgaagctcaagaaatatttggctatttgcctatagaagttcagcaatttatttcagaagaaatgaatgtaccattgacagagatatttggaatcgcgactttttactcacgtttcacattaaagccatccgggaagtataaaatcggcgtttgccttggcactgcttgttacgtaaaaggttctgcgatggtattagacaaattaaaagagaagcttggcataagcgtaggagacgtgacaggtgatggcaagttttcacttgaagcgactcgctgtttaggtgcttgcggtcttgcacctgtaatgatgataaacggagaagtttttggcagattgacacctgatgatgttgaagatatattgaagaaatttgattaa

SEQ ID NO: 4 - DNA, 387 bp, Thermoanaerobacterium saccharolyticum
ttgtgtcatggaggtgtaaatatgaaatctatagaggaattagaaaaaataagaaaagagacattggaaaaggtaaatcttcgtaaagatagaaacggcataagaattacggtcggcatggctacgtgtggtatagctgctggcgcaaggccagttatgatggctatattagatgagcttggcaagagaaatattacggatgtagttgttgctgagactggttgtatcggcatgtgcaaatatgagcctatggtagatgtttatgttcctggacaagaaaaagttacgtatataaaagttgatgaaaacaaggcaaggcagatagttgcggaacatgtagttaacggacatccgattaaagaatggactattagtagtgttgaataa

SEQ ID NO: 5 - DNA, 1716 bp, Thermoanaerobacterium saccharolyticum
atgagtgtcattaatttcaaagaagccaattgcagaaactgctataaatgcattagatattgccctgtaaaagcgataaaagtcaatgatgaacaggctgaaatcatagaatacaggtgcatagcatgcggaagatgcttaaatatctgtcctcagaatgcaaaaacagttagatcagacgtagaaagagttcaatcttttttaaataaaggagaaaaagttgctttcactgtagctccatcatatcctgctcttgttggacatgatggtgctttgaactttttaaaggctttaaaaagtttaggagccgaaatgatagttgagacatcagtaggtgctatgcttatatctaaggagtatgaaaggtattataatgatttgaaatatgacaatttgattactacttcatgtccatcggtaaattatttggttgaaaaatactaccctgatcttataaaatgccttgtgccagttgtatcgccgatggtggctgttggaagagctataaaaaatatacacggtgaaggtgtgaaagtcgtatttataggcccgtgccttgctaaaaaagcagagatgagcgattttagctgtgaaggcgctatagatgctgtattgacttttgaagaagtaatgaatttgtttaatacaaataaaataggtgttgaatgcacgaaagagaatttagaagatgttgactctgaaagccgatttaaattgtatccaatagagggcaaaaccatggattgcatggatgttgatttaaatttaagaaaatttatctctgtatcatcgatagaaaatgtgaaagatattttaaatgatttaagagctggcaatctacacggatattggatagaagctaatgcctgtgatggaggctgcatcaatggccctgcatttggaaagttagaaagtggtattgcaaaaagaaaagaagaagttataagctattctcgcatgaaagaaaggtttagcggtgatttcagcggcattaccgatttttccttagatttaagcagaaagtttattgatttaagtgatagatggaaaatgccaagcgagatggagataaaagagatattgtcgaagattggcaagttttctgtagaagacgaattgaattgcggtgcatgtggctatgacacttgcagggaaaaggctattgcagtctttaacggaatggcggaaccgtatatgtgcttgccatatatgagagggagggctgaaacgctgtctaatatcataataagttctactccaaacgctataattgcagttaataatgagtatgaaattcaagatatgaatagagcgtttgagaagatgtttttggtaaattcagccatggttaaaggtgaagatttatcgttgatctttgatatatctgattttgtagaggttattgaaaataagaaaagcatttttaataaaaaagtttcgtttaaaaattacggaatcatagcattggaaagcatctactatttggaagaatataaaattgccattggaatttttacagatataacaaagatggagaaacaaaaggagagcttctcaaagcttaaaagggaaaactaccaattggcgcagcaagtgatagatagacagatgaaggttgcacaagagatagcaagcttgttaggagaaacgactgcggagacaaaagtgatactgactaagatgaaagatatgctgttaaatcaaggtgatgatgaa

SEQ ID NO: 6 - DNA, 1158 bp, Thermoanaerobacterium saccharolyticum
atgagtcattacatcgatattgcacatgcatcattgaataaatacgatgaagaactgtgtggagatagtgttcaaataataagaaagaaagattatgcaatggcagttatggcagatggccttggcagcggtgttaaggcgaatattctatctactttgacaacgcgaatagtgtcaaaaatgttggatatgggttctgagctaagagatgttgtagaaacggtggctgagacattgccaatatgcaaagaaagaaatatagcgtattcaacatttactgttgtttctatatatggggacaatgctcatttagttgaatatgacaatccatcggttttttattttaagaatggtgtgcataagaaggtcgatagaaaatgtgttgaaataggtgataagaaaatctttgaaagcagcttcaaattggatttgaatgatgcgctgatagttgtatctgatggagtaattcatgcaggcgtaggagggatattaaatcttggttggcaatgggataatgttaaacaatatttatcaaaagtattggaagtttacagcgatgcatcagatatctgttcacaacttataacaacctgcaataatttgtacaaaaataggccaggcgatgatacaactgcaatagtgataaaagttaacgaatctaaaaaagttacggtaatggtaggaccgccgattttaaagaatatggatgaatgggttgttaaaaaactcatgaaaagtgaaggcttaaaggtagtatgtggtggtaccgctgcaaaaattgtaagcaggattttaaataaagacgtgattacatctaccgagtatattgatcctgatatacctccttatgcacatattgatgggattgatctggtgacagagggcgtattgactttaagaaagactgttgaaattttcaaagaatacatgaatgataaagactcaaatttgctgagattttcaaaaaaagatgctgcaactcgattatttaaaatcttaaattacgctactgacgtaaatttcttagtaggccaggctgtaaacagtgcccatcaaaatcctgattttccatccgatcttagaataaaggtcaggattgtggaagaacttataagcttattagagagattaaataaaaatgtggaagtaaattatttt

SEQ ID NO: 7 - DNA, 1485 bp, Thermoanaerobacterium saccharolyticum
atgttaaagtacgaggtgctttacaacgtagctaaattgacgcttgaagataggcttgaagatgaatacgacgaaataccttacgagataataccgggaacaaaaccgaggtttaggtgttgcgtgtataaggaaagggctataattgagcagagaactaaagtcgcaatggggaaaaatttaaagcgcactatgaaacatgcagttgacggtgaagagccgataattcaagttttagatattgcctgtgaggagtgtcctatcaaaaggtatcgtgtaactgaagcttgtagagggtgtattactcataggtgtacagaagtatgtccaaaaggagccataacgataataaacaaaaaggccaacatcgactacgacaagtgcatagagtgtggcaggtgcaaagatgcgtgtccatacaatgctatttctgacaatttgaggccgtgtattagatcttgttcagcaaaggccataactatggatgaagaattgaaagctgccataaattacgaaaaatgtacttcgtgtggtgcttgcacattggcatgtccattcggagccataaccgataagtcttatattgtagacattataagggcgattaagagcgggaaaaaagtttatgcattggtagcgccagccatagcatcccaatttaaggatgtaactgtaggacagataaaatctgctttaaaagaatttggatttgttgatgtgattgaagttgctcttggcgcagattttgtagctatggaagaagccaaagaattcagccataaaataaaagacataaaagtcatgacgagttcatgttgtcctgcatttgtggcacacataaagaaaagttatcctgagctatcgcaaaatatatcgacaactgtatctccaatgacagctatatcgaaatacatcaaaaaacacgatcctatggcagtgacagtatttataggtccatgtactgcaaagaaatcggaagtcatgagagatgatgtaaagggcataacggattttgccatgacatttgaagagatggttgctgtgttggatgcggcaaaaatagacatgaaagaacagcaagatgtggaagtggatgatgctacgctttttggaagaaagtttgcaagatctggaggcgtcttagaggctgtggttgaagccgttaaagaaataggcgcggatgttgaagtaaaccctgtagtatgcaatgggcttgatgaatgcaacaagacattgaaaataatgaaagctggcaaattgccaaacaattttatagaaggcatggcttgcatcggaggatgtataggcggtgcaggcgtaataaataacaatgtaaatcaggcaaaattggctgttaacaaatttggcgattcatcttaccataaaagcataaaagatagaatcagccaatttgatactgatgacgttgatttccatgttgacagcggtgaagatgagtcaagtgaaacatcgtttaaagaagct

SEQ ID NO: 8 - DNA, 243 bp, Thermoanaerobacterium saccharolyticum
atggttattactgtttgtgtaggaagttcatgccacttaaaaggttcctacgatgttataaacaaattaaaagaaatgattaaaaattacggtattgaggataaagtggagttgaaggctgacttttgcatgggcaattgtttaagggcggtttctgtaaaaattgatggcggtgcgtgtttatcaataaaaccaaatagcgttgagagattttttaaagaacatgttttaggtgaactaaaa

SEQ ID NO: 9 - protein, 587 aa, Thermoanaerobacterium saccharolyticum
MKGVQNMDKVRITIDGIPAEVPANYTVLQAAKYAKIEIPTLCYLEEINEIGACRLCVVEIKGVRNLQASCVYPVSDGMEIYTNTPRVREARRSNLELILSAHDRSCLTCVRSGNCELQDLSRKSGIDEIRFMGENIKYQKDESSPSIVRDPNKCVLCRRCVATCNNVQNVFAIGMVNRGFKTIVAPSFGRGLNESPCISCGQCIEACPVGAIYEKDHTKIVYDALLDEKKYVVVQTAPAVRVALGEEFGMPYGSIVTGKMVSALKRLGFDKVFDTDFAADLTIIEEGNELLKRLNEGGKLPMITSCSPGWINYCERYYPEFIDNLSTCKSPHMMMGAIIKSYFAEKEGIDPKDIFVVSIMPCTAKKYEIDRPQMIVDGMKDVDAVLTTRELARMIKQSGIDFVNLPDSEYDNPLGESSGAGVIFGATGGVMEAALRTVADIVEGKDIENFEYEEVRGLEGIKEAKIDIGGKEIKIAVANGTGNAKKLLDKIKNGEAEYHFIEVMGCPGGCIMGGGQPIHNPNEKDLVRKSRLKAIYEADKDLPIRKSHKNPMITKLYEEFLISPLGEKSHHLLHTTYSKKDLYPMND

SEQ ID NO: 10 - protein, 596 aa, Thermoanaerobacterium saccharolyticum
MLYRSHVMVCGGTGCTSSNSDRIAKCFEEEIANKGLDKEVQVVRTGCFGLCELGPVVVVYPEGVFYSCVKEEYVPEIVEEHLLKGRVVKKYLYGESVTEEGIKPLEETAFFKKQQRVALRNCGLINPEDIKEAIAFDGYKALAKVLTEMTPEEVINEIKKSGLRGRGGGGFPTGIKWEFAYNQKETPKYVVCNADEGDPGAFMDRSVLEGDPHSVLEAMAIAGYAIGANHGYIYVRAEYPLAVKRLQIAIDQAREYGLLGKNIFNTGFDFDIEIRLGAGAFVCGEETALLNSVMGKRGEPRPRPPFPAVKGVWEKPTIINNVETYANIPAIILNGAEWFASIGTEKSKGTKVFALGGKINNTGLVEIPMGTTLREIIFEIGGGIPNGKKFKAAQTGGPSGGCIPAEHLDTPIDYDSLLNIGSMMGSGGLIVMDEDNCMVDIAKFFLEFTVDESCGKCSPCRIGTRRMLELLNKITSGKGEEGDIEKLETLANSIKASSLCGLGQTAPNPVLSTIRYFRDEYEAHIKEKRCPAGVCQALLKFRIDPDKCKGCGICAKNCPTNAISGKVKQPHVIDQDKCIKCGTCMDKCPFDAIYKK

SEQ ID NO: 11 - protein, 160 aa, Thermoanaerobacterium saccharolyticum
MQAIYEKFSEENINKLKKVIDQLKDTDGSLIAVMNEAQEIFGYLPIEVQQFISEEMNVPLTEIFGIATFYSRFTLKPSGKYKIGVCLGTACYVKGSAMVLDKLKEKLGISVGDVTGDGKFSLEATRCLGACGLAPVMMINGEVFGRLTPDDVEDILKKFD

SEQ ID NO: 12 - protein, 128 aa, Thermoanaerobacterium saccharolyticum
MCHGGVNMKSIEELEKIRKETLEKVNLRKDRNGIRITVGMATCGIAAGARPVMMAILDELGKRNITDVVVAETGCIGMCKYEPMVDVYVPGQEKVTYIKVDENKARQIVAEHVVNGHPIKEWTISSVE

SEQ ID NO: 13 - protein, 572 aa, Thermoanaerobacterium saccharolyticum
MSVINFKEANCRNCYKCIRYCPVKAIKVNDEQAEIIEYRCIACGRCLNICPQNAKTVRSDVERVQSFLNKGEKVAFTVAPSYPALVGHDGALNFLKALKSLGAEMIVETSVGAMLISKEYERYYNDLKYDNLITTSCPSVNYLVEKYYPDLIKCLVPVVSPMVAVGRAIKNIHGEGVKVVFIGPCLAKKAEMSDFSCEGAIDAVLTFEEVMNLFNTNKIGVECTKENLEDVDSESRFKLYPIEGKTMDCMDVDLNLRKFISVSSIENVKDILNDLRAGNLHGYWIEANACDGGCINGPAFGKLESGIAKRKEEVISYSRMKERFSGDFSGITDFSLDLSRKFIDLSDRWKMPSEMEIKEILSKIGKFSVEDELNCGACGYDTCREKAIAVFNGMAEPYMCLPYMRGRAETLSNIIISSTPNAIIAVNNEYEIQDMNRAFEKMFLVNSAMVKGEDLSLIFDISDFVEVIENKKSIFNKKVSFKNYGIIALESIYYLEEYKIAIGIFTDITKMEKQKESFSKLKRENYQLAQQVIDRQMKVAQEIASLLGETTAETKVILTKMKDMLLNQGDDE

SEQ ID NO: 14 - protein, 386 aa, Thermoanaerobacterium saccharolyticum
MSHYIDIAHASLNKYDEELCGDSVQIIRKKDYAMAVMADGLGSGVKANILSTLTTRIVSKMLDMGSELRDVVETVAETLPICKERNIAYSTFTVVSIYGDNAHLVEYDNPSVFYFKNGVHKKVDRKCVEIGDKKIFESSFKLDLNDALIVVSDGVIHAGVGGILNLGWQWDNVKQYLSKVLEVYSDASDICSQLITTCNNLYKNRPGDDTTAIVIKVNESKKVTVMVGPPILKNMDEWVVKKLMKSEGLKVVCGGTAAKIVSRILNKDVITSTEYIDPDIPPYAHIDGIDLVTEGVLTLRKTVEIFKEYMNDKDSNLLRFSKKDAATRLFKILNYATDVNFLVGQAVNSAHQNPDFPSDLRIKVRIVEELISLLERLNKNVEVNYF

SEQ ID NO: 15 - protein, 495 aa, Thermoanaerobacterium saccharolyticum
MLKYEVLYNVAKLTLEDRLEDEYDEIPYEIIPGTKPRFRCCVYKERAIIEQRTKVAMGKNLKRTMKHAVDGEEPIIQVLDIACEECPIKRYRVTEACRGCITHRCTEVCPKGAITIINKKANIDYDKCIECGRCKDACPYNAISDNLRPCIRSCSAKAITMDEELKAAINYEKCTSCGACTLACPFGAITDKSYIVDIIRAIKSGKKVYALVAPAIASQFKDVTVGQIKSALKEFGFVDVIEVALGADFVAMEEAKEFSHKIKDIKVMTSSCCPAFVAHIKKSYPELSQNISTTVSPMTAISKYIKKHDPMAVTVFIGPCTAKKSEVMRDDVKGITDFAMTFEEMVAVLDAAKIDMKEQQDVEVDDATLFGRKFARSGGVLEAVVEAVKEIGADVEVNPVVCNGLDECNKTLKIMKAGKLPNNFIEGMACIGGCIGGAGVINNNVNQAKLAVNKFGDSSYHKSIKDRISQFDTDDVDFHVDSGEDESSETSFKEA

SEQ ID NO: 16 - protein, 81 aa, Thermoanaerobacterium saccharolyticum
MVITVCVGSSCHLKGSYDVINKLKEMIKNYGIEDKVELKADFCMGNCLRAVSVKIDGGACLSIKPNSVERFFKEHVLGELK

SEQ ID NO: 17 - DNA, 33 bp, Artificial
  synthetic oligonucleotides
ttactcgagaaactggtggaacatctggtggat

SEQ ID NO: 18 - DNA, 33 bp, Artificial
  Synthetic DNA
aagtctagataaatcgctccgacaggacatgct

SEQ ID NO: 19 - DNA, 34 bp, Artificial
  synthetic DNA
ctacaattggacttgcctatcagaaagtctcaca

SEQ ID NO: 20 - DNA, 33 bp, Artificial
  synthetic DNA
atagagctctcatgggagaaccagatgcaagta

SEQ ID NO: 21 - DNA, 31 bp, Artificial
  Synthetic DNA
atatctcgagctgtaattgtccttgatgacg

SEQ ID NO: 22 - DNA, 33 bp, Artificial
  synthetic DNA
atatctgcagcaggatatgatggagctacagtg

SEQ ID NO: 23 - DNA, 32 bp, Artificial
  synthetic DNA
atatgaattccatatatgagagggagggctga

SEQ ID NO: 24 - DNA, 29 bp, Artificial
  synthetic DNA
atatcggccgagtcgtttctcctaacaag

SEQ ID NO: 25 - DNA, 34 bp, Artificial
  synthetic DNA
tggatccgccatttattatttccttcctcttttc

SEQ ID NO: 26 - DNA, 27 bp, Artificial
  synthetic DNA
ttctagatggctgcaggtcgataaacc

SEQ ID NO: 27 - DNA, 34 bp, Artificial
  synthetic DNA
gcggatcccatgaacaaaaatataaaatattctc

SEQ ID NO: 28 - DNA, 31 bp, Artificial
  synthetic DNA
gcgaattccctttagtaacgtgtaactttcc